(19) United States
(12) Patent Application Publication
(10) Pub. No.: US 2022/0387272 A1
(43) Pub. Date: Dec. 8, 2022

(54) CLAY MASK FOR TREATING SKIN

(71) Applicant: Hani Hajomar, Cypress, CA (US)

(72) Inventor: Hani Hajomar, Cypress, CA (US)

(21) Appl. No.: 17/826,035

(22) Filed: May 26, 2022

Related U.S. Application Data
(60) Provisional application No. 63/193,825, filed on May 27, 2021.

(51) Int. Cl. A61K 8/25; A61K 8/02; A61Q 19/00; A61K 8/26; A61K 8/9789

(52) U.S. Cl. CPC A61K 8/25; A61K 8/0204; A61Q 19/00; A61K 8/26; A61K 8/9789; A61K 2800/524

(57) ABSTRACT

Described herein are clay masks for skin care and methods of making said masks. The mask may comprise Moroccan clay, rhassoul clay, Kaolin white clay, glycerin, aloe vera juice, rose water, and lavender hydrosol. Additionally, the mask may include fragrance or preservatives.

### CROSS-REFERENCE TO RELATED APPLICATION

This application claims priority to U.S. Provisional Patent Application No. 63/193,825, filed on May 27, 2021, which is incorporated herein by reference in its entirety.

### BACKGROUND

### Field

The present application relates to the field rinse-off masks for skin care.

### Description of the Related Technology

Masks for topical use generally are commercially distributed as a cream or a foam. The rinse-off mask is generally applied to clean skin of the body and more specifically the face. Face masks are generally worn from seconds to tens of minutes to allow for proper interaction with the body tissue or skin. Removal is accomplished using a water rinse, a wipe, or a combination.

Some prior masks cause skin irritation and/or overly dry the skin leading to uncomfortable, undesired use.

### SUMMARY

Some embodiments described herein relate to a clay mask comprising: about 25-29% Moroccan clay by volume; about 25-29% rhassoul clay by volume; about 11-15% Kaolin white clay by volume; about 7-11% glycerin by volume; about 18-22% aloe vera juice by volume; about 1-2% rose water by volume; and about 1-2% lavender hydrosol by volume.

In one embodiment, the mask comprises about 0.5-1.5% fragrance by volume. In another embodiment, the mask comprises about 0.1-1% preservatives by volume. In one embodiment, the preservatives comprise potassium sorbate. In another embodiment, the preservatives comprise sodium benzoate.

In some embodiments, the ratio of Moroccan clay to rhassoul clay to Kaolin white clay is about 2:2:1. In some embodiments, the mask is packaged in a less than an about 8 oz container. In some embodiments, the mask is packaged in an about 2 oz container.

Some embodiments described herein related to a method of making a clay mask comprising: about 25-29% Moroccan clay by volume; about 25-29% rhassoul clay by volume; about 11-15% Kaolin white clay by volume; about 7-11% glycerin by volume; about 18-22% aloe vera juice by volume; about 1-2% rose water by volume; and about 1-2% lavender hydrosol by volume; wherein the Moroccan clay, rhassoul clay, and Kaolin white clay are combined and initially separate from the other mask components.

One embodiment further comprises adding the glycerin to the combination of Moroccan clay, rhassoul clay, and Kaolin white clay. In another embodiment, one third of the glycerin is added at a time, and the combination is mixed until the glycerin is fully absorbed before subsequent glycerin is added.

In some embodiments, the aloe vera juice, rose water and lavender hydrosol are combined and initially separate from the other mask components. In some embodiments, fragrance is added to the combination. In some embodiments, preservatives are added to the combination. In some embodiments, the combination containing preservatives is added to the combination containing clays.

In some embodiments, fragrance is present in an amount of about 0.5-1.5% by volume. In some embodiments, preservatives are present in an amount of about 0.1-1% by volume.

Some embodiments relate to a clay mask comprising: about 45 mL Moroccan clay; about 45 mL rhassoul clay; about 22.5 mL Kaolin white clay; about 15 mL glycerin; about 33 mL aloe vera juice; about 2.5 mL rose water; and about 2.5 mL lavender hydrosol. Some embodiments further comprise about 1.5 mL fragrance. Some embodiments comprise about 0.866 mL preservatives.

### DETAILED DESCRIPTION

Embodiments of the innovations disclosed herein are particularly useful in removing grease, oils and/or toxins from skin, leaving skin with an increased degree of freshness and comfort. Moreover, the mask provides a soothing experience during wear and after removal. In some embodiments, use of the clay mask results in brighter skin and reduces acne caused by everyday grease. Other embodiments remove or reduce oiliness on skin without over-drying. Moreover, the mask may advantageously overall purify skin through cleanliness and removal of toxins layer on your skin. In some embodiments, use of the mask may lead to a reduction in the occurrence of acne.

### Definitions

The term “Moroccan clay” refers to a type of gentle clay used in the preparation of skincare products. For example, Moroccan clay from the vendor Bulk Apothecary may be used in the embodiments described herein.

The term “rhassoul clay” refers to a type of clay used in the preparation of skin care and bath products. For example, rhassoul clay from the vendor Bulk Apothecary may used in the embodiments described herein.

The term “kaolin white clay” refers to a clay used in the preparation of skincare products. It is also known as China Clay, and contains many minerals. For example, kaolin white clay from the vendor Bulk Apothecary may be used in the embodiments described herein.

The term “glycerin” refers to 1,2,3-propanetriol, a simple polyol compound.

The term “aloe vera juice” refers to a liquid made from the flesh of the aloe vera plant leaf.

The term “rose water” refers to an aqueous extract of rose petals.

The term “lavender hydrosol” refers to an aqueous extract of lavender flowers.

The term “fragrance” refers to a substance that gives off a sweet or pleasant odor.

The term “preservative” refers to a substance used to prevent materials from decaying.

The term “organic” as applied to the components described herein means that the component has received organic certification from the U.S. Department of Agriculture.

Preferably, embodiments of the innovation should be applied at end of an individual's day, and after removal, the individual should stay out of excessive sun exposure for 8-12 hours. However, beneficial results are likely to occur even without avoidance of sun exposure. Preferably, an individual applies the clay mask for 10-15 minutes and then removes through wash with water, soap and/or wipes.

In an embodiment, a user washes his or her face with cold water and then applies the clay mask using either fingers and/or a face brush applicator. In another embodiment, a user seeks to apply a relatively even layer throughout facial skin using a generous amount of cream.

Further embodiments of the innovation comprise a gel or cream. Moreover, the ingredients can be supplied premixed or in portions, and can be part of a single use or many use packaging. In some embodiments, the cream is packaged in 2 oz jars, preferably glass, along with a face brush applicator. The cream is pre-mixed in the jar. However, an artisan will recognize from the disclosure herein that squeezable tubes or bottles, plastic containers of nearly all types, or plastic makeup style jars or cans could be used to distribute the cream to customers. Such containers could be single use, many use, or sold by the volume or ounce.

According to one embodiment, a process of developing about 2 oz of the cosmetic clay mask includes the following steps.

Step 1:
Add about 45 mL of Moroccan clay;
Add about 45 mL of rhassoul clay;
Add about 22.5 mL of Kaolin white clay; and
Fully mix.

Moreover, an artisan will recognize the disclosure herein that larger or smaller batches can be made using similar or same percentages of the solutions or materials. Moreover, larger variability in a specific solution or material could be used. For example, an artisan will recognize the disclosure herein that embodiments of Moroccan clay could include about 5 mL to about 100 mL, and more preferably, embodiments of Moroccan clay could include about 20 mL to about 50 mL, or about 40 mL to about 50 mL, or about 43 mL to about 47 mL or about 44 mL to about 46 mL. Similarly, an artisan will recognize the disclosure herein that embodiments of Kaolin clay can include about 2 mL to about 100 mL, and more preferably, embodiments of Kaolin clay can include about 2 mL to about 40 mL, or about 20 mL to about 25 mL, or about 21 mL to about 24 mL, or about 22 mL to about 23 mL. Likewise, an artisan will recognize from the disclosure herein similar variability of the portion of rhassoul clay, that is, similar ranges and preferred ranges as described above for Moroccan clay.

Step 2:
Add about 15 mL of glycerin to the product of Step 1, mixing about 5 mL at a time until fully absorbed before adding a subsequent 5 mL portion; and
Mix until fully absorbed.

An artisan will recognize the disclosure herein that all the glycerin can be mixed at one time. In some cases, the glycerin can be mixed in increments (for example, 5 mL at a time as described above) until fully absorbed, until substantially absorbed, or until a majority of the glycerin is absorbed before adding a subsequent amount. Moreover, an artisan will recognize the disclosure herein that embodiments of glycerin could include about 0.25 mL to about 50 mL. In some embodiments, the total glycerin will be about 10 mL to about 20 mL, or about 13 mL to about 17 mL, or about 14 mL to about 16 mL.

Step 3:
Place the product of Step 2 aside;
Add about 33 mL of aloe vera juice to an empty container;
Add about 2.5 mL of rose water;
Add about 2.5 mL of lavender hydrosol;
Mix thoroughly;
Add about 1.5 mL of fragrance; and
Add preservatives

In an embodiment, the preservatives include about 0.433 mL of potassium sorbate and about 0.433 mL of sodium benzoate. In a preferred embodiment, the preservative should be around a minimum of 0.0025% of the total volume of the clay mask. Mix Step 2 fully, in an embodiment for about 10 to about 15 minutes, in other embodiments, anywhere from about 1 to about 20 minutes.

In an alternative embodiment, fragrance is omitted from Step 3. In yet another embodiment, preservatives are omitted from Step 3. In one embodiment, both fragrance and preservatives are omitted from Step 3.

In some embodiments, the preservatives may be one or more of: benzoic acid, sorbic acid, butylated hydroxyanisole, sodium nitrate, propionic acid, citric acid, sulfites, butylated hydroxytoluene, sodium nitrate, potassium nitrite, propylparaben, sulfur dioxide, propyl gallate, paraben, potassium nitrite, calcium sorbate, sodium metabisulphite, nisin, potassium benzoate, sodium sulfite, salt, and methylparaben.

In some embodiments, the fragrance may be one or more of: 3-Cyclohexene Carboxaldehyde, Alpha Amyl Cinnamic Alcohol, Anthemis Nobilis (Chamomile), Amyl Cinnamal, Anisyl Alcohol, Balsam of Peru (Myroxylon Pereirae), Benzaldehyde, Benzyl Alcohol, Benzyl Benzoate, Benzyl Cinnamate, Benzyl Salicylate, Butylphenyl Methylpropional, Cassia Oil, Chamomile (Anthemis Nobilis), Cinnamal, Cinnamic Alcohol, Cinnamic Aldehyde, Cinnamon Leaf Oil, Cinnamyl Alcohol, Citral, Citronellol, Clove Oil, Cologne, Coumarin, Ethylene Brassylate, Eugenol, Evernia Furfuracea, Evernia Prunastri, Farnesol, Geraniol, Herbal Extracts, Hexyl Cinnamal, Hydroxycitronellal, Hydroxylisohexyl 3-Cyclohexene Carboxaldehyde, Isoeugenol, Isomethyl Ionone, Limonene, Linalool, Lyral, Methyl 2-Octynoate, Oakmoss absolute, Perfume, Phenoxyethanol, Plant, flower, fruit, nut oils, and Yarrow (Achillea Millefolium).

In some embodiments, the composition is self-stable for 1, 2, 3, 4, 5, 6, 7, 8, 9, 10, 11, 12, 13, 14, 15, 16, 17, 18, 19, 20, 21, 22, 23, 24, 30, 36, 42, 48, 54, or 60 months. This self-stable composition is resistant to separation and mold growth.

An artisan will recognize the disclosure herein that embodiments of aloe vera juice could include about 0.5 mL to about 80 mL, and more preferably, about 15 mL to about 50 mL, or about 25 mL to about 40 mL, or about 30 mL to about 36 mL, or about 32 mL to about 34 mL. An artisan would further recognize from the disclosure herein that the rose water could be included in an amount from about 0.1 mL to 10 mL, and more preferably about 0.1 mL to about 5 mL, or about 1 mL to about 4 mL, or about 2 mL to about 3 mL. An artisan would recognize from the disclosure herein that the lavender hydrosol could be included in an amount from about 0.1 mL to 10 mL, and more preferably about 0.1 mL to about 5 mL, or about 1 mL to about 4 mL, or about 2 mL to about 3 mL. An artisan would further recognize from the disclosure herein that the fragrance could be included in an amount from about 0.1 mL to 10 mL, and more preferably about 0.1 mL to about 5 mL, or about 1 mL to 2 mL, or about 1.4 mL to about 1.6 mL. An artisan would recognize from the disclosure herein that the preservatives could be included in an amount from about 0.15 mL to 0.50 mL.

Step 4:
Slowly pour the product of Step 3 over the product of Step 2; and
Mix thoroughly until fully combined. In an embodiment, mix for about 10-15 minutes; and
Pour resulting mixture into distribution container. In an embodiment, the mix about fills a 2 ounce jar.

In an embodiment, one may add about 1-2% by volume of vitamin E oil. Also, an artisan will recognize other materials or solutions could be added to the mix, it could be heated, cooled, or repeatedly heated and cooled in any order.

The components of the topical compositions described herein may also be described in terms of percentage of total volume. It is important for the effectiveness of the composition that each component be present in an amount within the percentage ranges described herein. Including a component in an amount outside of the prescribed ranges may cause undesirable results, including but not limited to: redness when applied to the skin, mold growth within one month of preparing the composition, or separation of the composition into liquid and solid components within one month or preparing the composition.

In one embodiment, the composition comprises no more than three different types of clay. In an alternative embodiment, the percentages described herein are in terms of weight percent.

In other embodiments, the ratio of Moroccan clay to rhassoul clay to kaolin white clay is 1:1:1, 1:1:2, 1:1:3, 1:1:4, 1:1:5, 1:2:1, 1:2:2, 1:2:3, 1:2:4, 1:2:5, 1:3:1, 1:3:2, 1:3:3, 1:3:4, 1:3:5, 1:4:1, 1:4:2, 1:4:3, 1:4:4, 1:4:5, 1:5:1, 1:5:2, 1:5:3, 1:5:4, 1:5:5, 2:1:1, 2:1:2, 2:1:3, 2:1:4, 2:1:5, 2:2:1, 2:2:2, 2:2:3, 2:2:4, 2:2:5, 2:3:1, 2:3:2, 2:3:3, 2:3:4, 2:3:5, 2:4:1, 2:4:2, 2:4:3, 2:4:4, 2:4:5, 2:5:1, 2:5:2, 2:5:3, 2:5:4, 2:5:5, 3:1:1, 3:1:2, 3:1:3, 3:1:4, 3:1:5, 3:2:1, 3:2:2, 3:2:3, 3:2:4, 3:2:5, 3:3:1, 3:3:2, 3:3:3, 3:3:4, 3:3:5, 3:4:1, 3:4:2, 3:4:3, 3:4:4, 3:4:5, 3:5:1, 3:5:2, 3:5:3, 3:5:4, 3:5:5, 4:1:1, 4:1:2, 4:1:3, 4:1:4, 4:1:5, 4:2:1, 4:2:2, 4:2:3, 4:2:4, 4:2:5, 4:3:1, 4:3:2, 4:3:3, 4:3:4, 4:3:5, 4:4:1, 4:4:2, 4:4:3, 4:4:4, 4:4:5, 4:5:1, 4:5:2, 4:5:3, 4:5:4, 4:5:5, 5:1:1, 5:1:2, 5:1:3, 5:1:4, 5:1:5, 5:2:1, 5:2:2, 5:2:3, 5:2:4, 5:2:5, 5:3:1, 5:3:2, 5:3:3, 5:3:4, 5:3:5, 5:4:1, 5:4:2, 5:4:3, 5:4:4, 5:4:5, 5:5:1, 5:5:2, 5:5:3, 5:5:4, or 5:5:5.

It is also important for the effectiveness of the composition that the components be combined in the order described herein. Mixing the components together all at once, as opposed to stepwise, may cause undesirable results, including but not limited to: redness when applied to the skin, mold growth within one month of preparing the composition, or separation of the composition into liquid and solid components within one month or preparing the composition.

In an embodiment, the aloe vera juice, rose water, lavender hydrosol, fragrance, and preservatives are directly added to the mixture of remaining compounds without having been separately mixed first. In this embodiment, the resulting composition may separate into liquid and solid components within one week of preparation. In this embodiment, mold growth may visually appear within two weeks of preparation.

In an embodiment, one or more of the components of the composition are all-natural. In another embodiment, one or more of the components of the composition are organic. In yet another embodiment, one or more of the components of the composition are both all-natural and organic.

One embodiment comprises a kit of a clay mask composition as described herein and one or more facial wipes.

### EXAMPLES

### Example 1. Formulation of Mask Composition

Moroccan clay (45 mL), rhassoul clay (45 mL), and kaolin white clay (22.5) were combined and fully mixed. To this mixture, glycerin (15 mL) was added. The glycerin was added in 5 mL portions, and the resulting combination was mixed until each 5 mL portion was fully absorbed before a subsequent 5 mL portion was added. This resulted in the formation of a first mixture.

In a separate container, aloe vera juice (33 mL), rose water (2.5 mL), and lavender hydrosol (2.5 mL) were combined and fully mixed. Subsequently, fragrance (1.5 mL), potassium sorbate (0.433 mL), and sodium benzoate (0.433 mL) were added, and the combination was fully mixed, resulting in the formation of a second mixture.

The second mixture was slowly poured over the first mixture, and the resulting combination was stirred for 10 minutes. The resulting mask was transferred to air-tight jars for storage at room temperature.

### Example 2. Stability Studies (Prophetic)

The composition of Example 1 or a variant is stored in air-tight glass jars and the appearance of the composition is periodically observed over time.

In a jar containing the composition of Example 1, it is anticipated that the composition retains its original appearance, indicating that it is self-stable through 10 months. At 11 months it is anticipated that separation of the composition into liquid and solid components is visually observed.

A second jar contains a variation on the composition of Example 1, in which Moroccan clay comprises about 23% by volume, rhassoul clay comprises about 23% by volume, and kaolin white clay comprises about 9% by volume of the total composition. It is anticipated that this composition retains its original appearance, indicating that it is self-stable until 4 weeks pass. At 5 weeks it is anticipated that separation of the composition into liquid and solid components is visually observed.

### Example 3. Comparison of Masks Comprised of Differing Clays (Prophetic)

It is believed that skin care compositions formulated according to Example 1, when applied topically, will soften skin texture, reduce redness, and/or provide hydration. In order to test this, seven groups of ten subjects each are recruited for a study to compare different mask compositions. Each subject applies a layer of their assigned mask to the skin of the face. After 10 minutes, the subject removes the mask with a damp washcloth. Each subject repeats this routine every other day for seven days. At the end of the seven days, each subject provides an evaluation of their skin based on several parameters. Each group is assigned a mask identical to or similar to that described in Example 1, with and differences shown in Table 1, below. The only differences in the masks for each group are in the amounts or types of clay included in the compositions. The anticipated results of this experiment are shown in Table 2.

TABLE 1
Composition of Masks Assigned to Test Groups
Relative to the Mask of Example 1.

| Group Number | Clay Composition |
|---|---|
| 1 | Moroccan clay (112.5 mL) |
| 2 | rhassoul clay (112.5 mL) |
| 3 | Kaolin white clay (112.5 mL) |
| 4 | Moroccan clay (56.25 mL) and rhassoul clay (56.25 mL) |
| 5 | Moroccan clay (56.25 mL) and kaolin white clay (56.25 |
| | mL) |
| 6 | rhassoul clay (56.25 mL) and kaolin white clay (56.25 |
| | mL) |
| 7 | same as Example 1 |

TABLE 2
Anticipated Changes in Facial Skin of Test Subjects.

| Group | Subject | Softer Skin | Reduced Redness | Improved Hydration |
|---|---|---|---|---|
| 1 | 1 | x | | x |
| | 2 | | | x |
| | 3 | | | |
| | 4 | x | | |
| | 5 | x | | |
| | 6 | x | x | x |
| | 7 | | | |
| | 8 | | | |
| | 9 | | | |
| | 10 | | | |
| 2 | 1 | | | |
| | 2 | | | |
| | 3 | | | |
| | 4 | x | x | x |
| | 5 | | | |
| | 6 | | | |
| | 7 | x | x | x |
| | 8 | | | x |
| | 9 | | | |
| | 10 | x | | |
| 3 | 1 | x | x | x |
| | 2 | | | |
| | 3 | | | |
| | 4 | x | | |
| | 5 | x | x | x |
| | 6 | | | |
| | 7 | | | |
| | 8 | x | x | x |
| | 9 | x | | |
| | 10 | | | |
| 4 | 1 | | | |
| | 2 | x | | x |
| | 3 | x | x | x |
| | 4 | | | x |
| | 5 | | | |
| | 6 | x | x | |
| | 7 | x | x | x |
| | 8 | | | |
| | 9 | | | |
| | 10 | x | x | x |
| 5 | 1 | | x | x |
| | 2 | x | x | |
| | 3 | | | |
| | 4 | x | | x |
| | 5 | x | | x |
| | 6 | x | x | x |
| | 7 | | | |
| | 8 | x | x | |
| | 9 | | | |
| | 10 | | | |
| 6 | 1 | x | x | x |
| | 2 | | | |
| | 3 | | | |
| | 4 | x | | |
| | 5 | x | x | x |
| | 6 | x | x | x |
| | 7 | x | x | x |
| | 8 | | | |
| | 9 | | | |
| | 10 | | | |
| 7 | 1 | x | x | x |
| | 2 | x | x | x |
| | 3 | x | x | x |
| | 4 | x | | x |
| | 5 | | | |
| | 6 | x | x | x |
| | 7 | x | x | x |
| | 8 | x | x | x |
| | 9 | x | x | x |
| | 10 | x | | |

*An x indicates that it is believed that the subject will report this effect at the end of the experiment.

It is anticipated that half or less of the subjects will experience each of the three skin effects in groups 1-3, in which only one type of clay was used in each group. In this prophetic example, it is anticipated that 30-50% of the subjects of groups 1-3 report softer skin, 10-30% report reduced redness, and 30% report improved hydration.

Due to the complementary benefits of using more than one type of clay, it is anticipated that around half of the subjects will experience each of the three skin effects in groups 4-6, in which two types of clay were used in each group. In this prophetic example, it is anticipated that 50% of the subjects of groups 4-6 report softer skin, 40% report reduced redness, and 40-50% report improved hydration.

In view of the synergy between the three types of clays, it is anticipated that nearly all of the subjects in group 7 will experience each of the three skin effects. In this prophetic example, it is anticipated that between 70-100% of the subjects of group 7 report softer skin, between 70-100% report reduced redness, between 70-100% report improved hydration.

### Example 4. Use of Mask with Alternative Composition (Prophetic)

A variation on the composition of Example 1 is prepared, in which Moroccan clay comprises about 31% by volume, rhassoul clay comprises about 31% by volume, and kaolin white clay comprises about 17% by volume of the total composition. Ten subjects apply this composition to the skin of the face and let it sit for 10 minutes. After 10 minutes, each subject removes the composition with a damp washcloth. Once the composition is removed, the subjects report how their skin feels. Due to the increased proportions of each clay in the composition, it is anticipated that seven of the subjects will report increased redness of the skin, and eight subjects will report that their skin feels irritated.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate after reviewing the disclosure herein that in some embodiments, the actual steps taken in the processes illustrated and/or disclosed may differ from those shown in the FIGURES. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize, after reviewing the disclosure herein, that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The term “and/or” has similar meaning in that when used, for example, in a list of elements, the term “and/or” means one, some, or all of the elements in the list, but does not require any individual embodiment to have all elements.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

Values and ranges of values disclosed herein are examples and should not be construed as limiting. The values and ranges of values disclosed herein can be altered while gaining the advantages discussed herein. The listed ranges of values disclosed herein can include subsets of ranges or values which are part of this disclosure. Disclosed ranges of values or a single value for one feature can be implemented in combination with any other compatible disclosed range of values or value for another feature. For example, any specific value within a range of dimensions for one element can be paired with any specific value within a range of dimensions for another element.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Additionally, all publications, patents, and patent applications mentioned in this specification are herein incorporated by reference to the same extent as if each individual publication, patent, or patent application was specifically and individually indicated to be incorporated by reference.

What is claimed is:

1. A clay mask comprising:
about 25-29% Moroccan clay by volume;
about 25-29% rhassoul clay by volume;
about 11-15% Kaolin white clay by volume;
about 7-11% glycerin by volume;
about 18-22% aloe vera juice by volume;
about 1-2% rose water by volume; and
about 1-2% lavender hydrosol by volume.

2. The clay mask of claim 1, comprising about 0.5-1.5% fragrance by volume.

3. The clay mask of claim 1, comprising about 0.1-1% preservatives by volume.

4. The clay mask of claim 3, wherein the preservatives comprise potassium sorbate.

5. The clay mask of claim 3, wherein the preservatives comprise sodium benzoate.

6. The clay mask of claim 1, wherein the ratio of Moroccan clay to rhassoul clay to Kaolin white clay is about 2:2:1.

7. The clay mask of claim 1 packaged in a less than an about 8 oz container.

8. The clay mask of claim 7 packaged in an about 2 oz container.

9. A method of making a clay mask comprising:
about 25-29% Moroccan clay by volume;
about 25-29% rhassoul clay by volume;
about 11-15% Kaolin white clay by volume;
about 7-11% glycerin by volume;
about 18-22% aloe vera juice by volume;
about 1-2% rose water by volume; and
about 1-2% lavender hydrosol by volume;
wherein the Moroccan clay, rhassoul clay, and Kaolin white clay are combined and initially separate from the other mask components.

10. The method of claim 9, further comprising adding the glycerin to the combination of Moroccan clay, rhassoul clay, and Kaolin white clay.

11. The method of claim 10, wherein one third of the glycerin is added at a time, and the combination is mixed until the glycerin is fully absorbed before subsequent glycerin is added.

12. The method of claim 9, wherein the aloe vera juice, rose water and lavender hydrosol are combined and initially separate from the other mask components.

13. The method of claim 12, wherein fragrance is added to the combination.

14. The method of claim 13, wherein preservatives are added to the combination.

15. The method of claim 14, wherein the combination of claim 14 is added to the combination as described in claim 9.

16. The method of claim 15, wherein the fragrance is present in an amount of about 0.5-1.5% by volume.

17. The method of claim 16, wherein the preservatives are present in an amount of about 0.1-1% by volume.

18. A clay mask comprising:
about 45 mL Moroccan clay;
about 45 mL rhassoul clay;
about 22.5 mL Kaolin white clay;
about 15 mL glycerin;
about 33 mL aloe vera juice;
about 2.5 mL rose water; and
about 2.5 mL lavender hydrosol.

19. The clay mask of claim 18, comprising about 1.5 mL fragrance.

20. The clay mask of claim 18, comprising about 0.866 mL preservatives.
